(19) United States
(12) Patent Application Publication
(10) Pub. No.: US 2007/0165019 A1
(43) Pub. Date: Jul. 19, 2007

(54) Design Of systems For Improved Human Interaction

(76) Inventors: Kelly S. Hale, Orlando, FL (US); Leaha M. Reeves, Alexandria, VA (US); Kay M. Stanney, Oviedo, FL (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/457,061

(22) Filed: Jul. 12, 2006

Related U.S. Application Data
(60) Provisional application No. 60/698,531, filed on Jul. 12, 2005.

(51) Int. Cl. A61B 13/00; A61B 5/00; G06T 1/00

(52) U.S. Cl. 345/418; 600/558; 600/559

(57) ABSTRACT

A method for designing a human interface of a system includes establishing guidelines for avoiding sensory overload conditions of a human interacting with the system. The method also includes identifying an event associated with the system producing a potential sensory overload condition. The method further includes generating a human interface design solution based on the guidelines for modifying an operation of the system to help alleviate the potential sensory overload condition associated with the event. A prediction of a performance capability of a human subject interacting with the system may be made by determining a first parameter indicative of an intelligence of a human subject, determining a second parameter indicative of a multiple sensory input memory capacity of the human subject, and determining a third parameter indicative of an interactive monitoring capacity of the human subject and then using the parameters to generate an overall parameter indicative of a performance capacity.

### SPECIFIC DATA RELATED TO THE INVENTION

This application claims the benefit of U.S. Provisional Application No. 60/698,531 filed Jul. 12, 2005.

The U.S. Government has certain rights in this invention under contract number N61339-04-C-0037 awarded by NAVAIR.

### FIELD OF THE INVENTION

The present invention relates to human interface design, and, in particular, to optimizing a human interface of a system to improve a system operator's ability to process information provided via the system.

### BACKGROUND OF THE INVENTION

Today's military relies heavily on complex information systems, such as Command, Control, Communications, Computers, Intelligence, Surveillance, and Reconnaissance (C4ISR) systems, to gather information, monitor ongoing operations, and plan missions. In recent years, the amount of information an operator of such an information system must process and react to has risen dramatically. Consequently, the challenge of how to organize and present the vast amount of available data to operators so they can effectively and efficiently complete their missions is becoming increasingly more difficult. Traditionally, improving information processing capability to limit sensory and work overloads has focused on a layout of controls and information displays of the system and/or adding more operators to control and monitor the systems. However, sensory and work overload conditions are still encountered by operators of these systems.

### BRIEF DESCRIPTION OF THE DRAWINGS

FIG. 1 shows a flow chart for an example method for designing a human interface of an information system.

FIG. 2 shows a flow chart for an example method for predicting a performance capability of a human subject interacting with an information system.

### DETAILED DESCRIPTION OF THE INVENTION

The present invention is directed to design of systems for improved human interaction, for example, by ensuring that such systems present information in ways that reduce sensory and/or work overload conditions experienced by operators of the system. The inventors have realized that by providing systematic human interface design solutions for modifying information presentation of a system to better match demands with human perceptual and cognitive abilities, improved situational awareness and reduced sensory and work overload conditions of operators using such systems may be achieved.

In an embodiment, the invention automatically identifies, based on the events generated by a system, how to present information to an operator via different sensory channels, or multi-modally, to ensure critical tasks are perceived and comprehended accurately and acted upon in a timely fashion. For example, while using a visual light, i.e., a visual sensory channel, to indicate an imminent problem may be effective in a single display system, this type of presentation may not be effective when an operator is monitoring two or more visual displays at a time. Instead, an appropriate auditory and/or haptic alarm generated by the system may be implemented to ensure operators acknowledge and react to critical issues immediately and prevent further complications. Accordingly, when such a sensory overload situation is identified, one or more design solutions, such as a suggestion to provide an auditory or haptic alarm, may be automatically generated for alleviating the situation. By automatically providing human interface design solutions for presenting information more effectively, information display design may be simplified and design times may be decreased compared to conventional design techniques.

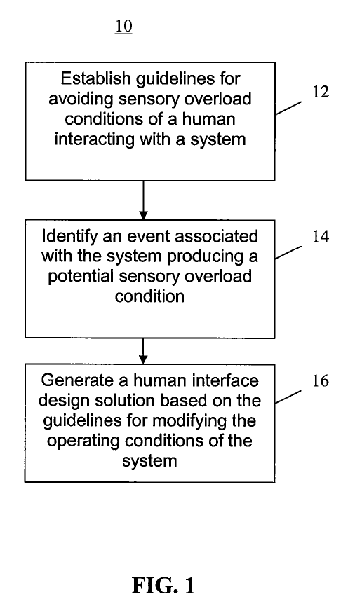

FIG. 1 shows a flow chart 10 of an example method for designing a human interface of a system. The method includes establishing guidelines for avoiding a sensory overload condition of a human interacting with an information system 12. Such guidelines may be derived from known guidelines for alleviating potential sensory overload conditions of a human interacting with an information systems via visual, auditory, haptic, and multi-modal sensory channels. A list of example guidelines for alleviating sensory overload conditions and associated rationale behind the guidelines is shown in Table 2:

TABLE 2
Example Guidelines for Remedying a Sensory Overload
Condition of a Human Interacting with an Information System

| | Sensory Channel | Guideline | Rationale |
|---|---|---|---|
| 1 | Visual | Avoid absolute | Individuals are much better at |
| | | judgment | distinguishing among different |
| | | (recognition | colors than at recognizing a |
| | | tasks) via color. | particular color. Therefore, avoid |
| | | | absolute judgment (“recognize”) |
| | | | tasks; design displays so that they |
| | | | require relative judgment |
| | | | (“distinguish”) tasks. |
| 2 | Visual | Design displays | Individuals are much better at |
| | | such that they | distinguishing among different |
| | | require relative | colors than at recognizing a |
| | | judgment via | particular color. Therefore, avoid |
| | | color | absolute judgment (“recognize”) |
| | | (differentiation | tasks; design displays so that they |
| | | tasks) | require relative judgment |
| | | | (“distinguish”) tasks. |
| 3 | Visual | Distribute | Visual information processing for |
| | | attention | color, shape, and motion are |
| | | amongst a range | distributed across distinct brain |
| | | of visual | regions. Leveraging these areas |
| | | characteristics of | may reduce visual cognitive |
| | | objects (i.e., | overload |
| | | shape, color, | |
| | | speed) to | |
| | | minimize | |
| | | cognitive | |
| | | workload | |
| 4 | Visual | Graphics are | Visual graphs are better when they |
| | | better than text | use spatial relations in ways that |
| | | or auditory | help a person ‘see’ relationships in |
| | | instructions for | the graphics. |
| | | communicating | |
| | | spatial | |
| | | information | |
| 5 | Visual | Make sure that | Studies have suggested that |
| | | the display can | approximately 8% of males and |
| | | be used without | less than 0.5% females have color |
| | | color (e.g., for | deficiencies. Therefore, when |
| | | color-blind | designing color displays, create |
| | | individuals) | elements that can be displayed |
| | | | without color. |
| 6 | Visual | Objects should | Visual processing are restricted to |
| | | be restricted to a | limited field of view of 180 degrees |
| | | field of 180° | horizontally and 130 degrees |
| | | horizontally and | vertically. |
| | | 130° vertically | |
| 7 | Visual | Present highest | Spatial tasks are best processed |
| | | priority spatial | via visual channels. Vision |
| | | task using visual | dominates spatial acuity since its |
| | | channel instead | acuity is about 1 min of arc as |
| | | of auditory | opposed to 1 deg for hearing. |
| | | channel. | |
| 8 | Visual | Present one task | To reduce visual overload and |
| | | at a time: Hold | optimize visual processing, present |
| | | lowest priority | highest priority visually. |
| | | task in cue until | |
| | | highest priority | |
| | | task is complete. | |
| 9 | Visual | Reaction time to | Visual cues require additional |
| | | visual stimuli | processing due to the complication |
| | | (180-200 msec) | of visual messages (i.e., shape, |
| | | is slower than | color, motion). |
| | | auditory (140-160 msec) | |
| | | and | |
| | | haptic (155 msec), | |
| | | thus it is | |
| | | best to use visual | |
| | | alerts and | |
| | | warnings only | |
| | | when these other | |
| | | modalities are | |
| | | loaded | |
| 10 | Visual | Text is better | For optimal processing, when |
| | | than speech for | conveying detailed and long |
| | | conveying | information visual text is better |
| | | detailed, long | than auditory speech since |
| | | information | audition tends to be transient. Due |
| | | | to its fleeting nature, speech will |
| | | | not be available for later review. |
| 11 | Visual | To examine | Visual acuity is optimal in the |
| | | object details, | center of the fovea, approximately |
| | | place object | two degrees of retina, Visual acuity |
| | | within foveal | is about 1 min of arc. |
| | | vision (central 2° | |
| | | of retina) | |
| 12 | Visual | Use animation to | Visual animation is critical to |
| | | demonstrate | understand a task. Animation is |
| | | sequential | best used as an interactive |
| | | actions in | technique for accuracy of decision |
| | | procedural tasks, | making tasks and should be used |
| | | simulate causal | when related to instructional |
| | | models of | objectives |
| | | complex system | |
| | | behavior, and | |
| | | explicitly | |
| | | represent | |
| | | invisible system | |
| | | functions and | |
| | | behaviors | |
| 13 | Visual | Use color to aid | Color coding is effective for visual |
| | | visual search by | search. The advantage of color is |
| | | making images | that it “catches the eye” more than |
| | | discriminable | other visual codes. |
| | | from one another | |
| 14 | Visual | Use congruent | The congruency effectiveness rule |
| | | pairings of color | suggests that certain congruent |
| | | and position to | combinations of cross-modal |
| | | reduce reaction | percepts will yield significantly |
| | | time | faster RT than incongruent |
| | | | combinations |
| 15 | Visual | Use congruent | The congruency effectiveness rule |
| | | pairings of pitch | suggests that certain congruent |
| | | and position to | combinations of cross-modal |
| | | reduce reaction | percepts will yield significantly |
| | | time | faster RT than incongruent |
| | | | combinations. RTs may be |
| | | | significantly shorter for congruent |
| | | | pairings of high pitch-high position |
| | | | (object placed above fixation on |
| | | | visual display) and low pitch-low |
| | | | position (object placed below |
| | | | fixation on visual display) pairings |
| | | | relative to RTs of incongruent |
| | | | pairings. A combination of pitch |
| | | | and color has been used to |
| | | | generate shorter RTs for congruent |
| | | | stimuli of white color-high pitch or |
| | | | black color-low pitch, as opposed |
| | | | to incongruent pairings (e.g., black |
| | | | color-high pitch). |
| 16 | Visual | Use flow charts | Visual graphs are better when they |
| | | to show | use spatial relations in ways that |
| | | relationships or | help a person ‘see’ relationships in |
| | | steps involved in | the graphics. |
| | | a process | |
| 17 | Visual | Use Gestalt | To increase visual information |
| | | Rules to increase | processing, enhance perceptual |
| | | users' | coding via Gestalt principles of |
| | | understanding of | proximity, similarity, and closure. |
| | | relationships | These principles include placing |
| | | between | related objects close together, |
| | | elements | enclosing related objects by lines |
| | | | or boxes, moving or changing |
| | | | related objects together, and |
| | | | ensuring related objects look alike |
| | | | (e.g., shape, color, size, |
| | | | topography). |
| 18 | Visual | Use motion to | To aid in visual direction, animate |
| | | enhance | visual images when object are not |
| | | detection of | in central foveal view or when |
| | | objects in the | display contains low illumination |
| | | periphery or | |
| | | overcome poor | |
| | | illumination | |
| 19 | Visual | Use numbered | Depict visual items with numbers |
| | | lists to show | to display order and relationships |
| | | groups of related | amongst objects. |
| | | items with a | |
| | | specific order | |
| 20 | Visual | Use tables, | Visual graphs are better when they |
| | | matrices, bar | use spatial relations in ways that |
| | | charts, pie charts | help a person ‘see’ relationships in |
| | | to help a person | the graphics. |
| | | ‘see’ | |
| | | relationships in | |
| | | the graphics. | |
| 21 | Visual | Use visual | Visual graphs are better when they |
| | | graphics for | use spatial relations in ways that |
| | | communicating | help a person ‘see’ relationships in |
| | | spatial | the graphics. |
| | | information | |
| 22 | Visual | Use visual text | For optimal processing, when |
| | | for conveying | conveying detailed and long |
| | | detailed, long | information visual text is best since |
| | | information. | it is permanent for operators to |
| | | | refer back to the message. |
| 23 | Auditory | A warning sound | |
| | | must be 15 dB | |
| | | above the | |
| | | threshold | |
| | | imposed by | |
| | | background | |
| | | noise to be heard | |
| | | clearly. | |
| 24 | Auditory | Add spatialized | |
| | | audio to aid | |
| | | identification of | |
| | | auditory verbal | |
| | | messages in | |
| | | noisy | |
| | | environments. | |
| 25 | Auditory | Auditory cues | |
| | | can be | |
| | | spatialized to | |
| | | indicate | |
| | | direction, | |
| | | location, and | |
| | | movement | |
| 26 | Auditory | Auditory icons | Auditory icons are vocal sounds |
| | | are useful when | that semantically relate |
| | | visual channel | environmental sounds to a given |
| | | overloaded | object (e.g., use the sound of a |
| | | | door opening to open a file). A |
| | | | listener's interpretation of the |
| | | | physical sound is considered a |
| | | | “sound symbol.” Auditory icons are |
| | | | useful in complex environments |
| | | | where users are visually |
| | | | overloaded; they are generally |
| | | | easy to learn and thus should be |
| | | | used for systems that require |
| | | | minimal training. |
| 27 | Auditory | If combining | |
| | | intensity | |
| | | differences with | |
| | | other auditory | |
| | | cues, use a | |
| | | minimum | |
| | | intensity of 10 dB | |
| | | above threshold | |
| | | and maximum | |
| | | intensity of 20 dB | |
| | | above threshold | |
| 28 | Auditory | If duration | |
| | | <500 ms, | |
| | | increase intensity | |
| | | to compensate | |
| | | for audibility | |
| | | (Sanders & | |
| | | McCormick, | |
| | | 1993) as sounds | |
| | | shorter than 500 ms | |
| | | may not be | |
| | | perceived. | |
| 29 | Auditory | Intensity should | |
| | | not be used | |
| | | alone for | |
| | | differentiating | |
| | | earcons | |
| 30 | Auditory | If pitch, register | |
| | | or rhythm are | |
| | | used alone to | |
| | | make absolute | |
| | | sound | |
| | | judgments, use a | |
| | | large difference | |
| | | between earcons | |
| | | (pitch: 125 Hz-5 kHz; | |
| | | register: 3 | |
| | | or more octaves; | |
| | | rhythm: different | |
| | | number of notes | |
| | | in each) | |
| 31 | Auditory | Keep auditory | Due to its transient nature, auditory |
| | | warning | information needs to be dealt with |
| | | messages simple | immediately. Only messages that |
| | | and short | will not be referred to at a later |
| | | | time should be conveyed via |
| | | | auditory displays. Auditory displays |
| | | | are thus preferred when |
| | | | information is simple and short. |
| | | | Limit recall of auditory items to |
| | | | about 3 or 4 elements. |
| 32 | Auditory | Keep auditory | |
| | | warning | |
| | | messages simple | |
| | | and short | |
| 33 | Auditory | Present one | |
| | | auditory task at a | |
| | | time: Hold lowest | |
| | | priority verbal | |
| | | task in cue until | |
| | | highest priority | |
| | | task is complete. | |
| 34 | Auditory | Present highest | Current understanding of Wickens' |
| | | priority verbal | Stimulus-Central Processing- |
| | | task using audio | Response compatibility (S-C-R) |
| | | instead of visual | schemes is that tasks demanding |
| | | input. | “verbal” WM, such as interpretation |
| | | | of system status, are thought to be |
| | | | best presented via audition (i.e., |
| | | | speech). |
| 35 | Auditory | Present low | |
| | | complexity, high | |
| | | priority | |
| | | information | |
| | | through the | |
| | | auditory channel. | |
| 36 | Auditory | Present lowest | To reduce visual overload and |
| | | priority spatial | optimize visual processing, present |
| | | task using | highest priority visually. Spatialized |
| | | spatialized audio | audio cues can be used to present |
| | | cues instead of | a lower priority task. |
| | | visual input | |
| 37 | Auditory | Present short | |
| | | lists using | |
| | | auditory channel | |
| | | instead of visual | |
| | | text. | |
| 38 | Auditory | Provide auditory | Providing auditory instructions will |
| | | rather than | minimize interference in the visual |
| | | textual | channel. |
| | | instructions when | |
| | | a listener is | |
| | | performing a | |
| | | visual task | |
| 39 | Auditory | Simulate human | |
| | | voices as much | |
| | | as possible when | |
| | | using speech | |
| 40 | Auditory | Speech is most | |
| | | effective for | |
| | | rapid, complex | |
| | | information | |
| 41 | Auditory | Use auditory | Auditory icons are vocal sounds |
| | | icons (with real | that semantically relate |
| | | world sounds) to | environmental sounds to a given |
| | | enhance their | object (e.g., use the sound of a |
| | | recognizability | door opening to open a file). A |
| | | | listener's interpretation of the |
| | | | physical sound is considered a |
| | | | “sound symbol.” Auditory icons are |
| | | | useful in complex environments |
| | | | where users are visually |
| | | | overloaded; they are generally |
| | | | easy to learn and thus should be |
| | | | used for systems that require |
| | | | minimal training. |
| 42 | Auditory | Use auditory | Due to its transient nature, auditory |
| | | messages if | information needs to be dealt with |
| | | dealing with time | immediately. Only messages that |
| | | relevant events, | will not be referred to at a later |
| | | continuously | time should be conveyed via |
| | | changing | auditory displays. Auditory displays |
| | | information, or | are thus preferred when |
| | | when requiring | information is simple and short. |
| | | immediate action | Auditory warning cues are superior |
| | | | to visual warnings and are better |
| | | | used when fast reaction time is |
| | | | essential (30 to 40 ms faster than |
| | | | vision). |
| 43 | Auditory | Use complex | Multiple encoding mechanisms for |
| | | sounds for | sound, such as frequency, |
| | | alarms | amplitude, and duration, can be |
| | | | used to aid in distinguishing among |
| | | | auditory signals). Auditory warning |
| | | | alerts are designed to use |
| | | | redundant dimensions such as |
| | | | pitch, timbre, and interruption |
| | | | rates. Auditory warning cues are |
| | | | superior to visual warnings and are |
| | | | better used when fast reaction time |
| | | | is essential (30 to 40 ms faster |
| | | | than vision). |
| 44 | Auditory | Use different | |
| | | voices for | |
| | | different interface | |
| | | elements | |
| 45 | Auditory | Use speech as a | |
| | | response method | |
| | | if user's hands | |
| | | are busy. | |
| 46 | Auditory | Use timbres with | Earcons use abstract, synthetic |
| | | multiple | sounds in structured combinations |
| | | harmonics to aid | to represent objects, interactions, |
| | | perception of | or operations. For example, the |
| | | critical items | size and type of a file may be |
| | | while avoiding | conveyed aurally (e.g., increase |
| | | masking | pitch to indicate a large file). Tones |
| | | | are good for communicating limited |
| | | | information sources (e.g., start or |
| | | | stop times) and may be used as |
| | | | complex sounds (i.e., using timbre |
| | | | as a grouping cue). Music may be |
| | | | used to combine sounds from |
| | | | various rhythms to provide an |
| | | | inherent structure that one can |
| | | | map to the structure of a dataset. |
| | | | Additionally, harmonic structures |
| | | | may be used to convey semantic). |
| 47 | Auditory | When playing | |
| | | sequential | |
| | | earcons, use a | |
| | | 0.1 s delay | |
| | | between them so | |
| | | listeners can tell | |
| | | when one | |
| | | finishes and the | |
| | | next commences | |
| 48 | Haptic | Gestures can be | Gestures should be intuitive and |
| | | used to | simple; avoid increasing user's |
| | | communicate | cognitive load with too numerous |
| | | meaningful | and/or complex. |
| | | information in | Avoid frequent, awkward or precise |
| | | isolation or in | gestures. |
| | | combination with | |
| | | speech and/or | |
| | | visual | |
| | | information | |
| 49 | Haptic | Tactile cues can | |
| | | be augmented by | |
| | | or substituted for | |
| | | visual tasks to | |
| | | aid localization | |
| 50 | Haptic | Vibratory cues | Reaction time to haptic stimuli is |
| | | can replace | 40 ms shorter than reaction time to |
| | | auditory cues for | visual (similar RT to auditory); thus |
| | | alerts/warnings | the haptic sense may serve as an |
| | | | effective warning signal. |
| 51 | Haptic | Add tactile cues | Tactile cues are effective at |
| | | to spatial tasks to | grabbing attention. Adding spatial |
| | | aid localization. | tactile cues to a visual scene may |
| | | | increase performance on spatial |
| | | | orientation tasks by grabbing |
| | | | attention towards visual display of |
| | | | interest. Tactile cues should not be |
| | | | used alone as they may not be |
| | | | ideal for quickly and precisely |
| | | | directing attention (although are |
| | | | effective at grabbing attention). |
| 52 | Haptic | Avoid | The motor system brain areas |
| | | unpredictable | include the brain stem, primary |
| | | tactile stimuli, as | motor cortex, associational cortex, |
| | | they tend to | basal ganglia, cerebellum, and the |
| | | increase cortical | premotor cortex and supplemental |
| | | activation | motor area (SMA) in the frontal |
| | | | lobe. Increased cortical activation |
| | | | across these areas has been |
| | | | documented when the stimulus to |
| | | | which one must respond is |
| | | | unpredictable. |
| 53 | Haptic | Present lowest | To reduce visual overload and |
| | | priority spatial | optimize visual processing, present |
| | | task using | highest priority visually. Spatialized |
| | | spatialized tactile | tactile cues can be used to present |
| | | cues instead of | a lower priority task. |
| | | visual input | |
| 54 | Haptic | Stimuli must be | |
| | | separated by at | |
| | | least 5.5 ms to | |
| | | be perceived as | |
| | | individual signals | |
| 55 | Haptic | Tactile cues can | Although visuo-spatial information |
| | | be augmented by | is thought to be best presented via |
| | | or substituted for | visual imagery, it could |
| | | visual tasks to | alternatively be conveyed via |
| | | aid localization | vibratory cues. For example, it has |
| | | | been demonstrated that the ability |
| | | | to substitute spatial information |
| | | | presented visually via tactile |
| | | | ‘vision.’ It has been demonstrated |
| | | | that tactile sensors can be |
| | | | effectively used to provide cues to |
| | | | resolve spatial disorientation in |
| | | | aviation environments. A Haptic |
| | | | driving navigation guidance system |
| | | | has been proposed that leverages |
| | | | a spatiotemporal illusion of |
| | | | movement across the back known |
| | | | as “sensory saltation,” which |
| | | | places three to six mechanical |
| | | | sensors that emit vibratory pulses |
| | | | with an interstimulus duration of 50 ms |
| | | | no greater than 10 cm apart |
| | | | along the back. |
| 56 | Haptic | Use force <4.7 N | |
| | | if sustained | |
| | | fingertip press | |
| | | required | |
| 57 | Haptic | Users should be | |
| | | able to actively | |
| | | search and | |
| | | survey the | |
| | | environment via | |
| | | touch and easily | |
| | | identify objects | |
| | | through physical | |
| | | interaction | |
| 58 | Multimodal | Add a tactile cue | Results show that reaction times |
| | | to direct | are faster when visual stimuli is |
| | | multimodal | presented following a tactile cue |
| | | interaction. | directing attention to the cued side. |
| | | | Multimodal cueing is thought to |
| | | | be based on external locations in |
| | | | space (posture-independent), not |
| | | | on a hemispheric (anatomical) |
| | | | model. |
| 59 | Multimodal | Add spatialized | It is known that the use of |
| | | audio to visual | spatialized audio in visual target |
| | | target detection | detection and presentation of 3D |
| | | tasks to | audio cues, emanating from the |
| | | decrease search | same spatial location as a visual |
| | | times | target, decreases search times. |
| | | | Auditory cues may be useful in |
| | | | visual target detection especially |
| | | | when a shift in gaze was required. |
| | | | A ‘frontal speech advantage’ has |
| | | | been demonstrated, where |
| | | | participants' driving performance |
| | | | increased when the focus of visual |
| | | | and auditory attention were from |
| | | | the same source (straight ahead) |
| | | | rather than when attention was |
| | | | divided between front (visual) and |
| | | | side (auditory) (e.g., as with a |
| | | | cellular phone ear piece). Thus, |
| | | | locate acoustic and visual stimuli |
| | | | within 160 of one another to |
| | | | produce greatest benefits. |
| 60 | Multimodal | Auditory cues | Audition aids in re-direction of gaze |
| | | added to a visual | by focusing a user's attention on |
| | | target detection | events in an environment. |
| | | task are | |
| | | beneficial, | |
| | | especially when | |
| | | a shift in gaze is | |
| | | required (e.g., in | |
| | | the periphery) | |
| 61 | Multimodal | Auditory signals | |
| | | can be coupled | |
| | | to haptic signals | |
| | | to increase | |
| | | reaction time | |
| 62 | Multimodal | Combine tactile | Tactile cues are effective at |
| | | cues with the | grabbing attention. Adding spatial |
| | | visual scene to | tactile cues to a visual scene may |
| | | improve | increase performance on spatial |
| | | performance on | orientation tasks by grabbing |
| | | spatial | attention towards visual display of |
| | | orientation tasks | interest. Tactile cues should not be |
| | | | used alone as they may not be |
| | | | ideal for quickly and precisely |
| | | | directing attention (although are |
| | | | effective at grabbing attention). |
| 63 | Multimodal | For navigation | Visual distance judgments from a |
| | | tasks, combine | virtual scene can be inaccurate. |
| | | visual | Adding additional cues, either |
| | | presentation with | haptic feedback or 3D audio, may |
| | | haptic feedback | create more accurate spatial |
| | | and/or 3D | knowledge. Ensure information |
| | | auditory cues to | from different modalities is close |
| | | indicate heading, | temporally or spatially. |
| | | location, distance | |
| 64 | Multimodal | Haptics can be | |
| | | coupled to | |
| | | auditory signals | |
| | | to increase | |
| | | reaction time | |
| 65 | Multimodal | Integrate speech | |
| | | output with other | |
| | | modalities (e.g., | |
| | | integrating a | |
| | | voice interface | |
| | | with a touch | |
| | | display) because | |
| | | current speech | |
| | | information may | |
| | | be very poor or | |
| | | difficult to use | |
| 66 | Multimodal | Pair speech with | Seech detection increasesmore |
| | | visual cues (i.e., | when visual cues (i.e., facial |
| | | facial | movements) areired with auditory |
| | | movements; lip | stimuli than when auditory stimuli |
| | | reading) to | were presented alone. |
| | | enhance speech | Designers must be cautious of |
| | | detection | cross-modal illusions that may |
| | | | occur when these two modalities |
| | | | are combined, such as the McGurk |
| | | | effect (what the observer hears is |
| | | | influenced by what he or she |
| | | | sees). To avoid incorrect |
| | | | perceptions and to activate |
| | | | necessary auditory cortices to |
| | | | ensure proper verbal processing |
| | | | when using visual-auditory |
| | | | displays to convey verbal |
| | | | information, it may be beneficial to |
| | | | use lip-synched animated agents |
| | | | (with valid speech mouth |
| | | | movements) or videotape a live |
| | | | speaker. |
| 67 | Multimodal | Precede visual | |
| | | information with | |
| | | an auditory alert | |
| | | tone to enhance | |
| | | perception. | |

Once overload-alleviating guidelines are established, the method may further include identifying an event associated with an information system producing a potential sensory overload condition for a human interacting with the system 14. In an aspect of the invention, identifying an event may include characterizing event information associated with the event. For example, the event information may be characterized according to a task category associated with event, such as a communication task required to be performed by the operator, a type of cognitive demand on the user associated with the task, a timing of the task, such as a frequency and/or duration of the task, a display and/or input mode used for the task, and/or a task priority associated with the event. An example task categorization list for a communication task in a shipborne C4ISR system is shown in Table 2 below:

TABLE 2
Example Task Categorization List for a Communication Task

| | | | | Type of | | | |
|---|---|---|---|---|---|---|---|
| Task | Task | | | Activity | | | |
| Category | Sub-Category | No. | Task | for Task | Duration | | Priority |
| COMM | Transmit | 1 | Weather | Speech | 3 | s | 1 |
| | Information | | Information - | | | | |
| | | | tactical | | | | |
| | | | significance | | | | |
| | | 2 | | Chat | 5 | s | 1 |
| | | 3 | Weather | Speech | 7 | s | 0 |
| | | | information - | | | | |
| | | | general forecast | | | | |
| | | | info | | | | |
| | | 4 | | Chat | 10 | s | 0 |
| | | 5 | Request/respond | Speech | 3 | s | 2 |
| | | | to CO | | | | |
| | | 6 | | Chat | 5 | s | 2 |
| | | 7 | Request/respond | Speech | 3 | s | 1 |
| | | | to CIC team | | | | |
| | | | member - | | | | |
| | | | tactical | | | | |
| | | 8 | | Chat | 5 | s | 1 |
| | | 9 | Request/respond | Speech | 3 | s | 0 |
| | | | to CIC team | | | | |
| | | | member - non- | | | | |
| | | | tactical | | | | |
| | | 10 | | Chat | 5 | s | 0 |
| | | 11 | Direct | Speech | 3 | s | 2 |
| | | | movement of | | | | |
| | | | entity (I.e., | | | | |
| | | | direct | | | | |
| | | | movement of | | | | |
| | | | ownship) | | | | |
| | | 12 | | Chat | 5 | s | 2 |
| | | 13 | Direct entity for | Speech | 7 | s | 2 |
| | | | information | | | | |
| | | | gathering | | | | |
| | | | mission (e.g., | | | | |
| | | | direct helo to | | | | |
| | | | obtain | | | | |
| | | | surveillance | | | | |
| | | | video of threat | | | | |
| | | | area) | | | | |
| | | 14 | | Chat | 10 | s | 2 |
| | | 15 | Request visual | Speech | 3 | s | 1 |
| | | | ID of target (I.e., | | | | |
| | | | from bridge of | | | | |
| | | | ship) | | | | |
| | | 16 | | Chat | 5 | s | 1 |
| | | 17 | Create/transmit | Paper | 10 | min | 2 |
| | | | daily intension | | | | |
| | | | message | | | | |
| | | 18 | Create/pass on | Paper | 15 | min | 1 |
| | | | turnover papers | | | | |
| | Receive | 19 | Weather | Audio | 3 | s | 1 |
| | Information | | Information - | | | | |
| | | | tactical | | | | |
| | | | significance | | | | |
| | | 20 | | Chat | 5 | s | 1 |
| | | 21 | Weather | Audio | 7 | s | 0 |
| | | | information - | | | | |
| | | | general forecast | | | | |
| | | | info | | | | |
| | | 22 | | Chat | 10 | s | 0 |
| | | 23 | Receive | Audio | 3 | s | 2 |
| | | | Request/information | | | | |
| | | | from CO | | | | |
| | | 24 | | Chat | 5 | s | 2 |
| | | 25 | Receive | Audio | 3 | s | 1 |
| | | | Request/information | | | | |
| | | | from CIC | | | | |
| | | | team member - | | | | |
| | | | tactical | | | | |
| | | 26 | | Chat | 5 | s | 1 |
| | | 27 | Receive | Audio | 3 | s | 0 |
| | | | Request/information | | | | |
| | | | from CIC | | | | |
| | | | team member - | | | | |
| | | | non-tactical | | | | |
| | | 28 | | Chat | 5 | s | 0 |
| | | 29 | Receive alert | Audio | 3 | s | 2 |
| | | | information | | | | |
| | | 30 | | Chat | 5 | s | 2 |
| | | 31 | Receive/review | Audio | 5 | min | 1 |
| | | | sitreps | | | | |
| | | 32 | | Chat | 5 | min | 1 |
| | | 33 | Receive/review | Audio | 5 | min | 1 |
| | | | daily intension | | | | |
| | | | message | | | | |
| | | 34 | | Chat | 5 | min | 1 |
| | | 35 | | paper | 5 | min | 1 |

After characterizing event information, such as by categorizing task information, the method may include assigning cognitive processing values to the events. The cognitive processing values may be assigned according to processing categories associated with the event activity, such as a stimulus category, a cognitive category, and/or a response category. The stimulus category may include incoming stimulus sensory channels, such as visual, auditory, and haptic stimuli. The cognitive category may include two cognition types, such as spatial cognition and verbal cognition type. The response category may include two response types, such as a motor or speech response. Respective cognitive processing values may be assigned to each of the categories that are used in receiving and responding to an input from an information system. In an aspect of the invention, cognitive processing values may be assigned according to according to known valuation techniques that rate cognitive processing workloads corresponding to processing categories on a subjective scale, such as a 7 point scale wherein 0 represents very low attention demand on an operator and 7 represent a very high attention demand on an operator. An example cognitive processing workload scoring scale for various sensory channels is shown in Table 3:

TABLE 3
Cognitive Processing Workload Scoring Scale

| CHAN- | | DEMAND |
|---|---|---|
| NEL | NATURE OF THE DEMAND DESCRIPTORS | VALUE |
| VISUAL | Visual Resource Not Used | 0.0 |
| | Visually Register/Detect (Detect Occurrence of | 3.0 |
| | Image) | |
| | Visually Inspect/Check (Discrete | 3.0 |
| | Inspection/Static Condition) | |
| | Visually Locate/Align (Selective Orientation) | 4.0 |
| | Visually Track/Follow (Maintain Orientation) | 4.4 |
| | Visually Discriminate (Detect Visual | 5.0 |
| | Differences) | |
| | Visually Read (Symbol) | 5.0 |
| | Visually Read (Text - 1-2 words) | 5.0 |
| | Visually Read (Text - sentence) | 5.8 |
| | Visually Scan/Search Monitor | 6.0 |
| | (Continuous/Serial Inspection) | |
| AUDI- | Auditory Resource Not Used | 0.0 |
| TORY | Detect/Register Sound (Detect Occurrence of | 1.0 |
| | Sound) | |
| | Orient to Sound (General Orientation/Attention) | 2.0 |
| | Interpret Semantic Content (Speech) Simple 3 | 3.0 |
| | (1-2 words) | |
| | Orient to Sound (Selective Orientation/Attention) | 4.2 |
| | Verify Auditory Feedback (Detect Occurrence of | 4.3 |
| | Anticipated Sound) | |
| | Interpret Semantic Content (Speech) Complex 6 | 6.0 |
| | (sentence) | |
| | Discriminate Sound Characteristics (Detect | 6.6 |
| | Auditory Differences) | |
| | Interpret Sound Patterns (pulse rates, etc.) | 7.0 |
| HAPTIC | Haptic resource not used | 0.0 |
| | Detect/Register Cue (Detect occurrence of cue) | 1.0 |
| | Orient to Cue (General Orientation/Attention) | 2.0 |
| | Interpret cue content (verbal information) | 3.0 |
| | Orient to Cue (Selective Orientation/Attention) | 4.2 |
| | Discriminate Vibration Characteristics | 6.6 |
| | Interpret Vibration Patterns | 7.0 |
| SPATIAL | Spatial Resource not used | 0.0 |
| | Automotive (Simple Association) | 1.0 |
| | Alternative Selection | 1.2 |
| | Motion perception and tracking (perceive and | 3.7 |
| | track the motion of other moving entities in the | |
| | environment) | |
| | Evaluation/Judgment concerning axes or | 4.6 |
| | translation or rotation (Visualization of space or | |
| | items in space, visualization of 3D objects or | |
| | environments, maps) | |
| | Rehearsal of spatial location | 5.0 |
| | Encoding/Decoding, Recall of spatial items | 5.3 |
| | Localization of self and/or others | 6.8 |
| | Interpolation/extrapolation of continuous | 7.0 |
| | functions | |
| VERBAL | Verbal Resource not used | 0.0 |
| | Automotive (Simple Association) | 1.0 |
| | Alternative Selection | 1.2 |
| | Signal/Sign Recognition of verbal items | 3.7 |
| | Evaluation/Judgment (Single aspect of general | 4.6 |
| | symbols, icons, and other figures translated into | |
| | linguistic items) | |
| | Rehearsal or verbal items (Review of steps or | 5.0 |
| | actions to be taken, includes checking against a | |
| | plan) | |
| | Encoding/Decoding, Recall of verbal items | 5.3 |
| | Evaluation/Judgment (multiple aspects including | 6.8 |
| | reasoning of abstract representations of real- | |
| | world information) | |
| | Estimation, Calculation, Conversion | 7.0 |
| | (Calculations of distance, time, ordering, priority) | |
| MOTOR | Motor Response not used | 0.0 |
| | Discrete Actuation (Button, Toggle, Trigger) | 2.2 |
| | Continuous Adjustive (Flight Control, Sensor | 2.6 |
| | Control) | |
| | Manipulative | 4.6 |
| | Discrete Adjustive (Rotary, Vertical Thumb | 5.5 |
| | Wheel, Lever Position) | |
| | Symbolic Production (Writing) | 6.5 |
| | Serial Discrete Manipulation (Keyboard) | 7.0 |
| SPEECH | Speech Response not used | 0.0 |
| | Simple (1-2 words) | 2.0 |
| | Complex (sentence) | 3.0 |

After assigning cognitive processing values to the events, such as by using the scoring values presented in Table 3, a predicted workload may be calculated for one or more events, such as by summing the cognitive processing values from the processing categories associated with the invention. For example, a predicted workload for an event may be calculated using Equation 1:
W_{T}ΣΣa_{t,i}+Σ[(n_{t,i}−1)cᵢᵢΣa_{t,i}]+ΣΣcᵢⱼΣ(a_{t,i}+a_{t,j}) 1.

wherein W_{T} is the total predicted workload at time T, a_{t,i} represents the attention (e.g., cognitive processing value) corresponding to a human interface channel i to perform a task t, n_{t,i} represents the number of tasks occurring at time t with attention being given to channel i, and cᵢⱼ represents a conflict between channels i and j. Accordingly, the first term represents a sum of an attention demand requirement placed on an operator during the event, the second term represents a penalty due to attention demand conflicts within the same channel, and the third term represents a penalty due to attention demand conflicts between different channels. It has been experimentally determined that a total predicted workload of 60 or more is indicative of potential operator sensory overload.

When a sensory overload condition for one or more events has been identified, the method may include generating a human interface design solution based on the guidelines for modifying the operating condition of the system to help alleviate the potential sensory overload condition associated with the event. The design solution may be based on the guidelines presented in Table 1 and knowledge of an operating condition of the system when an overload event has been identified. A system design solution may be suggested to alter the presentation of information by the system to reduce a likelihood of an operator experiencing sensory overload in response to the event. For example, a solution to a sensory overload condition caused by a stimulus to a primary sense, such as a visual cue, may be to generate a stimulus for a secondary sense, such as an auditory cue. Table 4 below includes example design solutions for sensory overload conditions that are based at least in part on the example guidelines presented in Table 2.

TABLE 4
Example Design Solutions for Sensory Overload Conditions

| OVERLOAD | Stimulus | Cognitive | Response | Duration | Priority | Interface | SOLUTION |
|---|---|---|---|---|---|---|---|
| Visual | 3.0 | | | | | | Use |
| channel | Visually | | | | | | congruent |
| overloaded | register/ | | | | | | pairings of |
| | detect | | | | | | color and |
| | (detect | | | | | | position to |
| | occurrence | | | | | | reduce |
| | of | | | | | | reaction time |
| | image) | | | | | | |
| Visual | 3.0 | | | | | | Use motion to |
| channel | Visually | | | | | | enhance |
| overloaded | register/ | | | | | | detection of |
| | detect | | | | | | objects in the |
| | (detect | | | | | | periphery or |
| | occurrence | | | | | | overcome |
| | of | | | | | | poor |
| | image) | | | | | | illumination |
| Visual | 3.0 | | | | High | | Precede |
| channel | Visually | | | | | | visual |
| overloaded | register/ | | | | | | information |
| | detect | | | | | | with an |
| | (detect | | | | | | auditory alert |
| | occurrence | | | | | | tone. |
| | of | | | | | | |
| | image) | | | | | | |
| Visual | 3.0 | | | | | | Use vibratory/ |
| channel | Visually | | | | | | tactile cues |
| overloaded | register/ | | | | | | for |
| | detect | | | | | | alerts/warning |
| | (detect | | | | | | |
| | occurrence | | | | | | |
| | of | | | | | | |
| | image) | | | | | | |
| Visual | 3.0 | | | | | | Auditory cues |
| channel | Visually | | | | | | added to a |
| overloaded | register/ | | | | | | visual target |
| | detect | | | | | | detection task |
| | (detect | | | | | | are beneficial, |
| | occurrence | | | | | | especially |
| | of | | | | | | when a shift in |
| | image) | | | | | | gaze is |
| | | | | | | | required (e.g., |
| | | | | | | | in the |
| | | | | | | | periphery) |
| Visual | 4.0 | | | | | | Combine |
| channel | Visually | | | | | | tactile cues |
| overloaded | locate/align | | | | | | with the visual |
| | (selective | | | | | | scene to |
| | orientation) | | | | | | improve |
| | | | | | | | performance |
| | | | | | | | on spatial |
| | | | | | | | orientation |
| | | | | | | | tasks |
| Visual | 4.4 | | | | | | For navigation |
| channel | Visually | | | | | | tasks, |
| overloaded | track/follow | | | | | | combine |
| | (maintain | | | | | | visual |
| | orientation) | | | | | | presentation |
| | | | | | | | with haptic |
| | | | | | | | feedback |
| | | | | | | | and/or 3D |
| | | | | | | | auditory cues |
| | | | | | | | to indicate |
| | | | | | | | heading, |
| | | | | | | | location, |
| | | | | | | | distance |
| Visual | 4.4 | | | | | | Distribute |
| channel | Visually | | | | | | attention |
| overloaded | track/ | | | | | | amongst a |
| | follow | | | | | | range of |
| | (maintain | | | | | | visual |
| | orientation) | | | | | | characteristics |
| | | | | | | | of objects |
| | | | | | | | (i.e., shape, |
| | | | | | | | color, speed) |
| | | | | | | | to minimize |
| | | | | | | | cognitive |
| | | | | | | | workload |
| Visual | 5.0 | | | | | | Auditory icons |
| channel | Visually | | | | | | are useful |
| overloaded | read | | | | | | when visual |
| | (symbol) | | | | | | channel |
| | | | | | | | overloaded |
| Visual | 5.0 | | | | | | Auditory icons |
| channel | Visually | | | | | | are useful |
| overloaded | discriminate | | | | | | when visual |
| | (detect | | | | | | channel |
| | visual | | | | | | overloaded |
| | differences) | | | | | | |
| Visual | 6.0 | | | | | | Distribute |
| channel | Visually | | | | | | attention |
| overloaded | scan/ | | | | | | amongst a |
| | search/ | | | | | | range of |
| | monitor | | | | | | visual |
| | (continuous/ | | | | | | characteristics |
| | serial | | | | | | of objects |
| | inspection) | | | | | | (i.e., shape, |
| | | | | | | | color, speed) |
| | | | | | | | to minimize |
| | | | | | | | cognitive |
| | | | | | | | workload |
| Visual | Any visual | | | | | | Add a tactile |
| channel | score >0 | | | | | | cue to direct |
| overloaded | | | | | | | multimodal |
| | | | | | | | interaction. |
| Visual | | 6.8 | | | | | Tactile cues |
| channel | | Spatial - | | | | | can be |
| overloaded | | localization | | | | | augmented by |
| | | of | | | | | or substituted |
| | | self | | | | | for visual |
| | | and/or | | | | | tasks to aid |
| | | others | | | | | localization |
| Visual | 2 visual/verbal tasks | | | | | | Present |
| channel | | | | | | | highest |
| overload | | | | | | | priority verbal |
| | | | | | | | task using |
| | | | | | | | audio instead |
| | | | | | | | of visual input. |
| Visual | 2 visual/verbal tasks | | | | | | Present one |
| channel | | | | | | | task at a time: |
| overload | | | | | | | Hold lowest |
| | | | | | | | priority task in |
| | | | | | | | cue until |
| | | | | | | | highest |
| | | | | | | | priority task is |
| | | | | | | | complete. |
| Visual | 4.0 | | | | | | Add |
| channel | Visually | | | | | | spatialized |
| overload | locate/align | | | | | | audio to visual |
| | (selective | | | | | | target |
| | orientation) | | | | | | detection |
| | | | | | | | tasks to |
| | | | | | | | decrease |
| | | | | | | | search times |
| Visual | 5.0 | | | | | | Use auditory |
| channel | Visually | | | | | | messages if |
| overload | read (text - | | | | | | dealing with |
| | 1-2 words) | | | | | | time relevant |
| | | | | | | | events, |
| | | | | | | | continuously |
| | | | | | | | changing |
| | | | | | | | information, or |
| | | | | | | | when |
| | | | | | | | requiring |
| | | | | | | | immediate |
| | | | | | | | action |
| Visual | 6.0 | | | | | | Pair speech |
| NOT | Auditory: | | | | | | with visual |
| overloaded | interpret | | | | | | cues (i.e., |
| | semantic | | | | | | facial |
| | content | | | | | | movements; |
| | (speech - | | | | | | lip reading) to |
| | sentence) | | | | | | enhance |
| | | | | | | | speech |
| | | | | | | | detection |
| Visual | 6.0 | | | | | | Pair speech |
| NOT | Auditory: | | | | | | with visual |
| overloaded | interpret | | | | | | cues (i.e., |
| | semantic | | | | | | facial |
| | content | | | | | | movements; |
| | (speech - | | | | | | lip reading) to |
| | 1-2 words) | | | | | | enhance |
| | | | | | | | speech |
| | | | | | | | detection |
| Auditory | 1.0 | | | | | | Vibratory cues |
| channel | Detect/ | | | | | | can replace |
| overload | Register | | | | | | auditory cues |
| | sound | | | | | | for alerts/ |
| | (detect | | | | | | warnings |
| | occurrence | | | | | | |
| | of sound) | | | | | | |
| Auditory | 2.0 | | | | | | Vibratory cues |
| channel | Orient to | | | | | | can replace |
| overload | sound | | | | | | auditory cues |
| | (general | | | | | | for alerts/ |
| | orientation/ | | | | | | warnings |
| | attention) | | | | | | |
| Auditory | 4.2 | | | | | | Vibratory cues |
| channel | Orient to | | | | | | can replace |
| overload | sound | | | | | | auditory cues |
| | (selective | | | | | | for alerts/ |
| | orientation/ | | | | | | warnings |
| | attention) | | | | | | |
| Auditory | 6.0 | | | | | | Never present |
| channel | Auditory: | | | | | | two verbal |
| overload | interpret | | | | | | messages at |
| | semantic | | | | | | the same time |
| | content | | | | | | Offload in |
| | (speech - | | | | | | time/pacing |
| | sentence) | | | | | | |
| Auditory | 6.0 | | | Long | | | Text is better |
| channel | Auditory: | | | | | | than speech |
| overload | Interpret | | | | | | for conveying |
| | Semantic | | | | | | detailed, long |
| | content | | | | | | information |
| | (speech - | | | | | | |
| | sentence) | | | | | | |
| Auditory | 6.0 | | | | | | Keep auditory |
| channel | Interpret | | | | | | warning |
| overload | semantic | | | | | | messages |
| | content | | | | | | simple and |
| | (speech- | | | | | | short |
| | sentence) | | | | | | |
| Auditory | 7.0 | | | | | | Use auditory |
| channel | Interpret | | | | | | icons (with |
| overload | Sound | | | | | | real world |
| | Patterns | | | | | | sounds) to |
| | (pulse | | | | | | enhance their |
| | rates, etc). | | | | | | recognizability |
| Auditory | 7.0 | | | | | | Use timbres |
| channel | Interpret | | | | | | with multiple |
| overload | Sound | | | | | | harmonics to |
| | Patterns | | | | | | aid perception |
| | (pulse | | | | | | of critical |
| | rates, etc). | | | | | | items while |
| | | | | | | | avoiding |
| | | | | | | | masking |
| Spatial | Auditory | 6.8 | | | | | Use visual |
| channel | score >0 | Spatial - | | | | | graphics for |
| overloaded | for spatial | localization | | | | | communicating |
| | task | of self | | | | | spatial |
| | | and/or | | | | | information |
| | | others | | | | | |
| Spatial | Auditory | 6.8 | | | | | Present |
| channel | score >0 | Spatial - | | | | | highest |
| overloaded | for spatial | localization | | | | | priority spatial |
| | task | of self | | | | | task using |
| | | and/or | | | | | visual channel |
| | | others | | | | | instead of |
| | | | | | | | auditory |
| | | | | | | | channel. |
| Spatial | Auditory | 6.8 | | | | | Add tactile |
| channel | score >0 | Spatial - | | | | | cues to spatial |
| overloaded | for spatial | localization | | | | | tasks to aid |
| | task | of self | | | | | localization. |
| | | and/or | | | | | |
| | | others | | | | | |
| Spatial | Visual score | 6.8 | | | | | Tactile cues |
| channel | >0 for | Spatial - | | | | | can be |
| overloaded | spatial task | localization | | | | | augmented by |
| | | of self | | | | | or substituted |
| | | and/or | | | | | for visual |
| | | others | | | | | tasks to aid |
| | | | | | | | localization |
| Spatial | 2 visual/spatial tasks | | | | | | Present one |
| channel | | | | | | | task at a time: |
| overload + visual | | | | | | | Hold lowest |
| channel | | | | | | | priority spatial |
| overload | | | | | | | task in cue |
| | | | | | | | until highest |
| | | | | | | | priority task is |
| | | | | | | | complete. |
| Spatial | 2 visual/spatial tasks | | | | | | Present |
| channel | | | | | | | lowest priority |
| overload + visual | | | | | | | spatial task |
| channel | | | | | | | using |
| overload | | | | | | | spatialized |
| | | | | | | | audio cues |
| | | | | | | | instead of |
| | | | | | | | visual input |
| Spatial | 2 visual/spatial tasks | | | | | | Present |
| channel | | | | | | | lowest priority |
| overload + visual | | | | | | | spatial task |
| channel | | | | | | | using |
| overload | | | | | | | spatialized |
| | | | | | | | tactile cues |
| | | | | | | | instead of |
| | | | | | | | visual input |
| Verbal | 2 visual/verbal tasks | | | | | | Present |
| channel | | | | | | | highest |
| overload | | | | | | | priority verbal |
| | | | | | | | task using |
| | | | | | | | audio instead |
| | | | | | | | of visual input. |
| Verbal | visual/verbal tasks | | | | | | Present one |
| channel | | | | | | | task at a time: |
| overload | | | | | | | Hold lowest |
| | | | | | | | priority verbal |
| | | | | | | | task in cue |
| | | | | | | | until highest |
| | | | | | | | priority task is |
| | | | | | | | complete. |
| Verbal | 5.0 | | | <5 s | | | Present short |
| channel | Visually | | | | | | lists using |
| overload | read (text - | | | | | | auditory |
| | 1-2 words) | | | | | | channel |
| | | | | | | | instead of |
| | | | | | | | visual text. |
| Verbal | 7.0 | | | >5 s | | | Use visual |
| channel | Auditory | | | | | | text for |
| overload | Interpret | | | | | | conveying |
| | semantic | | | | | | detailed, long |
| | content | | | | | | information. |
| | (speech - | | | | | | |
| | sentence) | | | | | | |
| Verbal | 7.0 | | | | | | Add |
| channel | Auditory | | | | | | spatialized |
| overload | Interpret | | | | | | audio to aid |
| | sound | | | | | | identification |
| | patterns | | | | | | of auditory |
| | (pulse | | | | | | verbal |
| | rates, etc.) | | | | | | messages in |
| | | | | | | | noisy |
| | | | | | | | environments. |
| Motor | | | | | | | Use speech |
| channel | | | | | | | as a response |
| overload | | | | | | | method if |
| | | | | | | | user's hands |
| | | | | | | | are busy. |
| Speech | | | | | | | |
| channel | | | | | | | |
| overload | | | | | | | |
| | Any visual | | | | | | Use Gestalt |
| | score >0; | | | | | | Rules to |
| | not visually | | | | | | increase |
| | read (text) | | | | | | users' |
| | | | | | | | understanding |
| | | | | | | | of |
| | | | | | | | relationships |
| | | | | | | | between |
| | | | | | | | elements |
| | 3.0 | | | Short | High | | Reaction time |
| | Visually | | | | | | to visual |
| | register/detect | | | | | | stimuli (180-200 msec) |
| | (detect | | | | | | is |
| | occurrence | | | | | | slower than |
| | of image) | | | | | | auditory (140-160 msec) |
| | | | | | | | and haptic |
| | | | | | | | (155 msec), |
| | | | | | | | thus it is best |
| | | | | | | | to use visual |
| | | | | | | | alerts and |
| | | | | | | | warnings only |
| | | | | | | | when these |
| | | | | | | | other |
| | | | | | | | modalities are |
| | | | | | | | loaded |
| | 3.0 | | | | | One | To examine |
| | Visually | | | | | task not | object details, |
| | inspect/check | | | | | on main | place object |
| | (discrete | | | | | visual | within foveal |
| | inspection/static | | | | | interface | vision (central |
| | condition) | | | | | | 2° of retina; |
| | 5.0 | | | | | | Use animation |
| | Visually | | | | | | to |
| | read | | | | | | demonstrate |
| | (symbol) | | | | | | sequential |
| | | | | | | | actions in |
| | | | | | | | procedural |
| | | | | | | | tasks, |
| | | | | | | | simulate |
| | | | | | | | causal models |
| | | | | | | | of complex |
| | | | | | | | system |
| | | | | | | | behavior, and |
| | | | | | | | explicitly |
| | | | | | | | represent |
| | | | | | | | invisible |
| | | | | | | | system |
| | | | | | | | functions and |
| | | | | | | | behaviors |
| | 5.0 | Verbal | | | | | Provide aural |
| | Visually | task + second | | | | | rather than |
| | read (text - | task | | | | | textual |
| | 1-2 words) + second | | | | | | instructions |
| | visual task | | | | | | when a |
| | | | | | | | listener is |
| | | | | | | | performing a |
| | | | | | | | visual task |
| | 5.0 | | | Short | | | Speech is |
| | Visually | | | | | | most effective |
| | read (text - | | | | | | for rapid, |
| | 1-2 words) | | | | | | complex |
| | | | | | | | information |
| | 5.8 | Spatial - | | | | | Graphics are |
| | Visually | encoding/ | | | | | better than |
| | read - text | decoding, | | | | | text or |
| | (sentence) | recall | | | | | auditory |
| | | of spatial | | | | | instructions |
| | | items | | | | | for |
| | | | | | | | communicating |
| | | | | | | | spatial |
| | | | | | | | information |
| | 5.0 | | | | | | Avoid |
| | Visually | | | | | | absolute |
| | discriminate | | | | | | judgment |
| | (detect | | | | | | (recognition |
| | visual | | | | | | tasks) via |
| | differences) | | | | | | color |
| | 5.0 | | | | | | Make sure |
| | Visually | | | | | | that the |
| | discriminate | | | | | | display can be |
| | (detect | | | | | | used without |
| | visual | | | | | | color (e.g., for |
| | differences) | | | | | | color-blind |
| | | | | | | | individuals) |
| | 5.0 | | | | | | Design |
| | Visually | | | | | | displays such |
| | discriminate | | | | | | that they |
| | (detect | | | | | | require |
| | visual | | | | | | relative |
| | differences) | | | | | | judgment via |
| | | | | | | | color |
| | | | | | | | (differentiation |
| | | | | | | | tasks) |
| | 5.0 | | | | | | Use color to |
| | Visually | | | | | | aid visual |
| | discriminate | | | | | | search by |
| | (detect | | | | | | making |
| | visual | | | | | | images |
| | differences) | | | | | | discriminable |
| | | | | | | | from one |
| | | | | | | | another |
| | 5.0 | | | | | | Use |
| | Visually | | | | | | numbered |
| | discriminate | | | | | | lists to show |
| | (detect | | | | | | groups of |
| | visual | | | | | | related items |
| | differences) | | | | | | with a specific |
| | | | | | | | order |
| | 5.0 | | | | | | Use flow |
| | Visually | | | | | | charts to |
| | discriminate | | | | | | show |
| | (detect | | | | | | relationships |
| | visual | | | | | | or steps |
| | differences) | | | | | | involved in a |
| | | | | | | | process |
| | 5.0 | | | | | | Use tables, |
| | Visually | | | | | | matrices, bar |
| | discriminate | | | | | | charts, pie |
| | (detect | | | | | | charts for |
| | visual | | | | | | appropriate |
| | differences) | | | | | | uses . . . |
| | 1.0 | | | | | | Use |
| | Auditory: | | | | | | congruent |
| | Detect/Register | | | | | | pairings of |
| | sound | | | | | | pitch and |
| | (detect | | | | | | position to |
| | occurrence | | | | | | reduce |
| | of sound) | | | | | | reaction time |
| | 1.0 | | | | | | Keep auditory |
| | Auditory: | | | | | | warning |
| | Detect/Register | | | | | | messages |
| | sound | | | | | | simple and |
| | (detect | | | | | | short |
| | occurrence | | | | | | |
| | of sound) | | | | | | |
| | 1.0 | | | | | | Use complex |
| | Auditory: | | | | | | sounds for |
| | Detect/Register | | | | | | alarms |
| | sound | | | | | | |
| | (detect | | | | | | |
| | occurrence | | | | | | |
| | of sound) | | | | | | |
| | 1.0 | | | <500 ms | | | If duration |
| | Auditory: | | | | | | <500 ms, |
| | Detect/Register | | | | | | increase |
| | sound | | | | | | intensity to |
| | (detect | | | | | | compensate |
| | occurrence | | | | | | for audibility |
| | of sound) | | | | | | as sounds |
| | | | | | | | shorter than |
| | | | | | | | 500 ms may |
| | | | | | | | not be |
| | | | | | | | perceived. |
| | 2.0 | | | | High | | Haptics can |
| | Auditory: | | | | | | be coupled to |
| | orient to | | | | | | auditory |
| | sound | | | | | | signals to |
| | (general | | | | | | increase |
| | orientation/ | | | | | | reaction time |
| | attention) | | | | | | |
| | 2.0 | | | | | | Auditory cues |
| | Auditory: | | | | | | can be |
| | orient to | | | | | | spatialized to |
| | sound | | | | | | indicate |
| | (general | | | | | | direction, |
| | orientation/ | | | | | | location, and |
| | attention) | | | | | | movement |
| | 3.0 | | | | | | Simulate |
| | Auditory: | | | | | | human voices |
| | interpret | | | | | | as much as |
| | semantic | | | | | | possible when |
| | content | | | | | | using speech |
| | (speech - | | | | | | |
| | 1-2 words) | | | | | | |
| | 3.0 | | | | | | Use different |
| | Auditory: | | | | | | voices for |
| | interpret | | | | | | different |
| | semantic | | | | | | interface |
| | content | | | | | | elements |
| | (speech - | | | | | | |
| | 1-2 words) | | | | | | |
| | 4.2 | | | | High | | Haptics can |
| | Auditory: | | | | | | be coupled to |
| | orient to | | | | | | auditory |
| | sound | | | | | | signals to |
| | (selective | | | | | | increase |
| | orientation/ | | | | | | reaction time |
| | attention) | | | | | | |
| | 4.2 | | | | | | Auditory cues |
| | Auditory: | | | | | | can be |
| | orient to | | | | | | spatialized to |
| | sound | | | | | | indicate |
| | (selective | | | | | | direction, |
| | orientation/ | | | | | | location, and |
| | attention) | | | | | | movement |
| | 6.0 | | | | | | Simulate |
| | Auditory: | | | | | | human voices |
| | interpret | | | | | | as much as |
| | semantic | | | | | | possible when |
| | content | | | | | | using speech |
| | (speech - | | | | | | |
| | sentence) | | | | | | |
| | 6.0 | | | | | | Use different |
| | Auditory: | | | | | | voices for |
| | interpret | | | | | | different |
| | semantic | | | | | | interface |
| | content | | | | | | elements |
| | (speech - | | | | | | |
| | sentence) | | | | | | |
| | 6.0 | 5.3 | | | | | Graphics are |
| | Auditory: | Spatial - | | | | | better than |
| | interpret | encoding/ | | | | | text or |
| | semantic | decoding, | | | | | auditory |
| | content | recall | | | | | instructions |
| | (speech - | of spatial | | | | | for |
| | sentence) | items | | | | | communicating |
| | | | | | | | spatial |
| | | | | | | | information |
| | 6.6 | | | | | | A warning |
| | Auditory: | | | | | | sound must |
| | discriminate | | | | | | be 15 dB |
| | sound | | | | | | above the |
| | characteristics | | | | | | threshold |
| | (detect | | | | | | imposed by |
| | auditory | | | | | | background |
| | differences) | | | | | | noise to be |
| | | | | | | | heard clearly. |
| | 6.6 | | | | | | If pitch, |
| | Auditory: | | | | | | register or |
| | discriminate | | | | | | rhythm are |
| | sound | | | | | | used alone to |
| | characteristics | | | | | | make |
| | (detect | | | | | | absolute |
| | auditory | | | | | | sound |
| | differences) | | | | | | judgments, |
| | | | | | | | use a large |
| | | | | | | | difference |
| | | | | | | | between |
| | | | | | | | earcons |
| | | | | | | | (pitch: 125 Hz-5 kHz; |
| | | | | | | | register: 3 or |
| | | | | | | | more octaves; |
| | | | | | | | rhythm: |
| | | | | | | | different |
| | | | | | | | number of |
| | | | | | | | notes in each) |
| | 6.6 | | | | | | Intensity |
| | Auditory: | | | | | | should not be |
| | discriminate | | | | | | used alone for |
| | sound | | | | | | differentiating |
| | characteristics | | | | | | earcons |
| | (detect | | | | | | |
| | auditory | | | | | | |
| | differences) | | | | | | |
| | 6.6 | | | | | | If combining |
| | Auditory: | | | | | | intensity |
| | discriminate | | | | | | differences |
| | sound | | | | | | with other |
| | characteristics | | | | | | auditory cues, |
| | (detect | | | | | | use a |
| | auditory | | | | | | minimum |
| | differences) | | | | | | intensity of 10 dB |
| | | | | | | | above |
| | | | | | | | threshold and |
| | | | | | | | maximum |
| | | | | | | | intensity of 20 dB |
| | | | | | | | above |
| | | | | | | | threshold |
| | 6.6 | | | | | | When playing |
| | Auditory: | | | | | | sequential |
| | discriminate | | | | | | earcons, use |
| | sound | | | | | | a 0.1 s delay |
| | characteristics | | | | | | between them |
| | (detect | | | | | | so listeners |
| | auditory | | | | | | can tell when |
| | differences) | | | | | | one finishes |
| | | | | | | | and the next |
| | | | | | | | commences |
| | 1.0 | | | | | | Avoid |
| | Haptic: | | | | | | unpredictable |
| | detect/register | | | | | | tactile stimuli, |
| | cue | | | | | | as they tend |
| | (detect | | | | | | to increase |
| | occurrence | | | | | | cortical |
| | of cue) | | | | | | activation |
| | 2.0 | | | | High | | Auditory |
| | Haptic: | | | | | | signals can be |
| | orient to | | | | | | coupled to |
| | cue | | | | | | haptic signals |
| | (general | | | | | | to increase |
| | orientation/ | | | | | | reaction time |
| | attention) | | | | | | |
| | 4.2 | | | | High | | Auditory |
| | Haptic: | | | | | | signals can be |
| | orient to | | | | | | coupled to |
| | cue | | | | | | haptic signals |
| | (selective | | | | | | to increase |
| | orientation/ | | | | | | reaction time |
| | attention) | | | | | | |
| | 6.6 | | | | | | Stimuli must |
| | Haptic: | | | | | | be separated |
| | discriminate | | | | | | by at least 5.5 ms |
| | vibration | | | | | | to be |
| | characteristics | | | | | | perceived as |
| | | | | | | | individual |
| | | | | | | | signals |
| | | Verbal | | <5 s | High | | Present low |
| | | 5.3 or | | | | | complexity, |
| | | less | | | | | high priority |
| | | | | | | | information |
| | | | | | | | through the |
| | | | | | | | auditory |
| | | | | | | | channel. |
| | | Spatial | | <5 s | High | | Present low |
| | | 1.2 or | | | | | complexity, |
| | | less | | | | | high priority |
| | | | | | | | information |
| | | | | | | | through the |
| | | | | | | | auditory |
| | | | | | | | channel. |
| | | Verbal | | >5 s | Low | | Present high |
| | | 6.8 or | | | | | complexity, |
| | | more | | | | | low priority |
| | | | | | | | information |
| | | | | | | | through the |
| | | | | | | | visual |
| | | | | | | | channel. |

The above described method may be used, for example, when redesigning a system. The method may used to modify an existing system to improve information presentation, such as by assessing overload conditions, generating a solution, redesigning the system according to the suggested solutions. In another aspect, a on-line approach may be used to modify a system, for example, based on overload condition identified during use and then implementing a design solution while the system is operating.

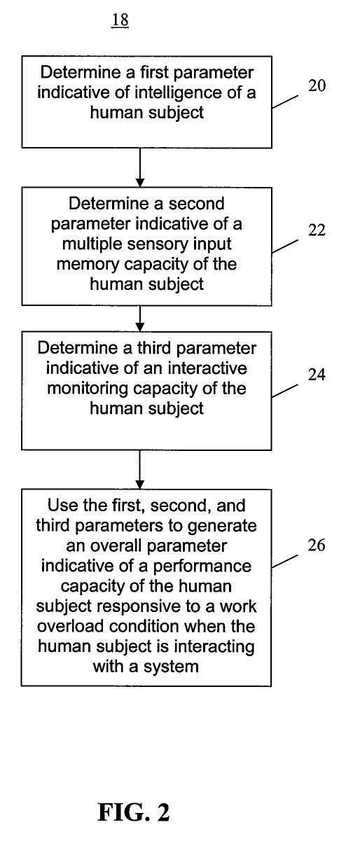

In another aspect of the invention, a method is provided for predicting a performance capability of a human subject interacting with a system, for example, to identify operators having superior information processing abilities that may be best suited to operate complex information systems. FIG. 2 shows an example flow chart 18 of a method for predicting a performance capability of a human subject interacting with an information system. The method includes determining a first parameter indicative of intelligence of a human subject 20 such as by using a general intelligence, or intelligence quotient (IQ), test to assess a subject's mental ability. For example, a test such as Raven's Progressive Matrices, may be used to test a subject to determine a first parameter, such as a test score to be used in predicting the subject's information processing abilities.

The method may also include determining a second parameter indicative of a multiple sensory input memory, or working memory, capacity of the human subject 22. Working memory reflects a limited capacity of the human brain for allowing temporary storage and manipulation of information for complex tasks as comprehension, learning, and reasoning. Accordingly, a working memory capacity assessment may be used to rate a subject's reasoning, decision making and planning abilities. In an embodiment of the invention, a method for determining a working memory capacity may include assessing a subject's ability to process multiple streams of information coming from different sensory sources, such as by testing a subject's memory of information presented to the subject via different sensory channels. The method may include presenting a subject with one or more visual, text, picture, speech, spatialzed tones, and/or spatialzed haptic cue stimuli and then assessing the subject's ability to recall the stimuli presented and/or the types of stimuli remembered. A score based on the above working memory capacity test may be used as the second parameter for predicting the subject's information processing abilities.

The method may also include determining a third parameter indicative of an interactive monitoring capacity of the human subject 24, such as by testing a subject's ability to dynamically interact with a simulated system to predict the subject's performance within a desired operational environment. For example, an interactive monitoring test similar to the known Federal Aviation Administration's (FAA) Air Traffic Selection and Training exam may be used to test a subject to determine the third parameter, such as a test score, to be used in predicting the subject's information processing abilities.

While each of the above described tests may separately provide an indication of an operator's ability to perform in certain environment, the inventors have realized that a combination of the tests may provide a better characterization of a subject's performance capability with regard to information processing. Accordingly, the method further includes using the first, second, and third parameters to generate an overall parameter indicative of a performance capacity of the subject 26, for example, responsive to a work overload condition when the human subject is interacting with a system. It has been experimentally determined that the overall parameter derived using the above method provides a better indication of information processing capability than any one of the tests separately.

Based on the foregoing specification, the invention may be implemented using computer programming or engineering techniques including computer software, firmware, hardware or any combination or subset thereof, wherein the technical effect is to generate design solutions for designing a human interface of an information system and generate a performance parameter for use in predicting a performance capability of a human subject interacting with a system. Any such resulting program, having computer-readable code means, may be embodied or provided within one or more computer-readable media, thereby making a computer program product, i.e., an article of manufacture, according to the invention. The computer readable media may be, for instance, a fixed (hard) drive, diskette, optical disk, magnetic tape, semiconductor memory such as read-only memory (ROM), etc., or any transmitting/receiving medium such as the Internet or other communication network or link. The article of manufacture containing the computer code may be made and/or used by executing the code directly from one medium, by copying the code from one medium to another medium, or by transmitting the code over a network.

One skilled in the art of computer science will easily be able to combine the software created as described with appropriate general purpose or special purpose computer hardware, such as a microprocessor, to create a computer system or computer sub-system embodying the method of the invention. An apparatus for making, using or selling the invention may be one or more processing systems including, but not limited to, a central processing unit (CPU), memory, storage devices, communication links and devices, servers, I/O devices, or any sub-components of one or more processing systems, including software, firmware, hardware or any combination or subset thereof, which embody the invention.

Although several embodiments of the present invention and its advantages have been described in detail, it should be understood that mutations, changes, substitutions, transformations, modifications, variations, and alterations can be made therein without departing from the teachings of the present invention, the spirit and scope of the invention being set forth by the appended claims.

What is claimed is:

1. A method for designing a human interface of a system comprising:
establishing guidelines for avoiding sensory overload conditions of a human interacting with a system;
identifying an event associated with the system producing a potential sensory overload condition; and
generating a human interface design solution based on the guidelines for modifying an operation of the system to help alleviate the potential sensory overload condition associated with the event.

2. The method of claim 1, wherein the design solution comprises an instruction to change a presentation of information by the system effective to reduce a likelihood of an operator experiencing sensory overload in response to the event.

3. The method of claim 1, wherein the design solution comprises an instruction to convert a first sense stimulus resulting in the event into a second sense stimulus effective to reduce a likelihood of an operator experiencing sensory overload in response to the event.

4. The method of claim 3, wherein the first sense stimulus is directed to at least one of a visual, an auditory, and a haptic sense.

5. The method of claim 1, wherein identifying an event comprises characterizing event information associated with the event.

6. The method of claim 5, wherein characterizing event information comprises organizing the event information into one or more task categories.

7. The method of claim 6, wherein the task categories comprise at least one of a task type, a type of cognitive demand on the user for the task, a timing of the task, a display mode used for the task, an input mode required by the task, and a priority of the task.

9. The method of claim 1, further comprising assigning a cognitive processing value to the event.

10. The method of claim 9, wherein the cognitive processing value is assigned according to at least one of an attention demand requirement placed on an operator during the event, an attention demand conflict in a same sensory channel of the system, and an attention demand conflict in different sensory channels of the system.

11. The method of claim 1, further comprising using the human interface design solution to modify the operation of the system while the system is being used.

12. A computer system having a processor, a memory, and an operating environment, the computer system configured for executing the method recited in claim 1.

13. A computer-readable medium having computer-executable instructions for performing the method recited in claim 1.

14. A method for predicting a performance capability of a human subject interacting with a system comprising:
determining a first parameter indicative of an intelligence of a human subject;
determining a second parameter indicative of a multiple sensory input memory capacity of the human subject;
determining a third parameter indicative of an interactive monitoring capacity of the human subject; and
using the first, second, and third parameters to generate an overall parameter indicative of a performance capacity of the human subject responsive to a work overload condition when the human subject is interacting with a system.

15. The method of claim 14, wherein determining a second parameter comprises assessing the subject's ability to remember information provided to the subject via different sensory channels.

16. The method of claim 15, wherein assessing the subject's ability comprises:
presenting a plurality of different sensory stimuli to the subject; and
testing the subject's ability to recall the stimuli presented.

17. The method of claim 15, wherein the stimuli are selected from the group consisting of visual, text, picture, speech, spatialzed tones, and spatialzed haptic cue stimuli.

18. The method of claim 14, further comprising using the overall parameter to identify operators having a desired performance capacity.

19. A computer system having a processor, a memory, and an operating environment, the computer system configured for executing the method recited in claim 14.

20. A computer-readable medium having computer-executable instructions for performing the method recited in claim 14.
